(19) United States
(12) Patent Application Publication
(10) Pub. No.: US 2021/0396733 A1
(43) Pub. Date: Dec. 23, 2021

(54) LAYERED NANOSTRUCTURE WITH NANOCRACKS AND NANOPORES AND METHOD OF PRODUCING THE SAME

(71) Applicant: Zedna AB, Täby (SE)

(72) Inventors: Valentin DUBOIS, Stockholm (SE); Niklaus FRANK, Täby (SE); Göran STEMME, Lidingö (SE)

(73) Assignee: Zedna AB, Täby (SE)

(21) Appl. No.: 17/272,295

(22) PCT Filed: Aug. 30, 2019
(86) PCT No.: PCT/SE2019/050816

§ 371 Date: Feb. 27, 2021

(30) Foreign Application Priority Data
Aug. 31, 2018 (SE) 1800168-5

(51) Int. Cl. G01N 33/487; C23F 1/02

(52) U.S. Cl. CPC G01N 33/48721; B82Y 15/00; C23F 1/02

(57) ABSTRACT

A layered nanostructure including a crack-forming layer with a first notch and a second notch provided in the crack-forming layer and the first notch is disclosed. A nanocrack is provided between the first notch and the second notch. Strain release in the tensilly stressed crack-forming layer is utilized in the layered nanostructure so that the nanocrack is very uniformed and well controlled with a width that may be below 10 nm. Nanopore devices including crossing nanocracks may be provided.

### FIELD OF THE INVENTION

The present invention relates to a layered nanostructure, a detecting device based on a plurality of layered nanostructures and method of producing such. Particularly the invention relates to a layered structure wherein the nanostructure is formed by one or more well defined gaps formed utilizing strain release in a tensilly stressed layer.

### BACKGROUND OF THE INVENTION

Nanostructures such as cracks (i.e. gaps) and pores (i.e. openings) that are patterned on wafers are building blocks of many important applications in stochastic sensing of single molecules in nanopores based on ionic or quantum tunneling currents. However, obtaining patterned surfaces on a nanometer-level in thin films that are flat on an atomic level is not possible using conventional masking (e.g. optical lithography) or etching (e.g. plasma etching) techniques. Obtaining structural reproducibility from pore-to-pore on a nanometer-level, ideally atomic level, is highly desirable in most nanopore applications, but extremely challenging.

US 2018/03772653 discloses crack structures that comprises a substrate and a spacer material layer on the substrate having at least one open space. There is a layer of one or more selected material(s) provided on the spacer material, the material layer being patterned to exhibit a crack-defined gap between two cantilevering parts extending across said open space, wherein the width of the crack-defined gap is predetermined by the length of the cantilevering parts and by the built-in stress. Optionally said cantilevering parts are collapsed onto the substrate. The crack is preferably less than 100 nm wide, more preferably less than 3 nm wide thereby forming a tunnelling junction. It further discloses a method of making a crack structure on a substrate, the crack structure being usable as a tunneling junction structure in a nanogap device, including the controlled fracture or release of a patterned layer under built-in stress, thereby forming elements separated by nanogaps, or crack-junctions. The width of the crack is controlled by locally release-etching the film at a notched bridge patterned in the film. The built in stress contributes to forming the crack and defining of the width of the crack. The nanocracks can be used for tunneling devices in combination with nanopores for DNA, RNA, or peptides sequencing.

Nanocracks in general, and nanocracks in single crystalline layers in particular, circumvent the limitations of conventional patterning, and enable reproducible patterning of surfaces with near atomic-level accuracy through e.g. crystallographically oriented channeling cracks. Nanocracks formed by the crack-junction methodology disclosed in US 2018/03772653 have the additional advantage of forming with very high speed and in a highly parallel fashion through crack formation. Moreover, the fabrication of stress concentration structures that induce and localize crack formation is compatible with conventional wafer-scale patterning. Thus, nanocracks formed by the crack-junction methodology disclosed in US 2018/03772653 can form in a highly parallel fashion on wafer-scale.

A nanopore structure can for example be used for sensing of single molecules, molecular sieveing/filtration, protein detection and analysis, energy conversion etc. Today, nanopores are either biological nanopores (lipid-based), solid-state nanopores or nanogap electrodes embedded in solid-state membranes. Biological nanopores suffer from several problems such as limited shelf life and limitations when it comes to the length of the pore. In solid-state nanopores these problems are in principle circumvented. However, at present the most promising technology for solid-state nanopores is to drill holes in two dimensional materials, e.g. graphene, to form a pore. This is very time consuming and cannot be performed in series, only one-at-a-time.

I. Fernandez-Martinez et al. Nanotechnology 19 (2008) 275302 discloses a nanogap fabrication process using strained epitaxial III-V beams, that are potentially highly reproducible, allow for parallel fabrication and nanogap size control. The stress causing the breakage is built into the beam.

H. Arjmandi-Tash et al. Adv. Mater. 2018, 30, 1703602 discloses nanocapillaries of zero depth by dissolving two superimposed and crossing metallic nanorods, molded in polymeric slabs.

Prior art represents significant advances in the area of technology. However it is still a challenge to provide nanostructures such as nanocracks with the high degree of precision and reproducibility required to provide detecting devices, for example, on an industrial scale. Especially for cracks with a width of around, or less than, 10 nm, a gap size that would be highly advantageous in for example DNA characterization. More complex geometries such as nanopores, remain even more challenging.

### SUMMARY OF THE INVENTION

The object of the invention is to provide a layered nanostructure, that solves, or at least reduces, the problems with the prior art. This is achieved by the multi-layered crack structure as defined in claim 1, the nanopore structure as defined in claim 10, and the method as defined in claim 11.

The layered nanostructure according to the invention comprises a crack-forming layer with an upper and lower surface and a membrane at least partly in contact with and at least partly covering the upper or lower surface of the crack-forming layer. A first notch and a second notch are provided in and extending through the crack-forming layer and the first notch is provided at a distance from the second notch. A nanocrack is provided between the first notch and the second notch and divides the crack-forming layer into a first and second side relative the nanocrack. A through opening is arranged in the membrane and positioned so that at least a portion of the nanocrack is aligned with the opening of the membrane. A continuous opening is formed by the first notch, the second notch and nanocrack in the crack-forming layer and the continuous opening is surrounded by a continuous structure formed in the crack-forming layer which connects the first and the second sides of the nanocrack.

According to one aspect of the invention the diameter of the first notch and the diameter of the second notch, respectively, is between 0.1 to 5 times the length of the nanocrack.

According to one aspect of the invention the membrane is provided with at least one opening which at least in one direction is aligned with the nanocrack so that at least a portion of the nanocrack is positioned over or under the opening in the membrane.

According to one aspect of the invention the first notch in a cross-section in the plane of the crack-forming layer comprises a blunt section and a sharp-edged section. The sharp-edge section is positioned in a part of the first notch facing the second notch. The nanocrack extends from the sharp-edged section of the first notch to the second notch.

According to one aspect of the invention also second notch in a comprises of a blunt section and a sharp-edged section and the nanocrack extends from the sharp-edge section of the first notch to the sharp-edge section of the second notch. The nanocrack may extend in the direction representing the shortest distance between the first notch and the second notch.

According to one aspect of the invention the first notch forms a and the second notch (102b) forms, in a cross-section in the plane of the crack-forming layer, smooth continuous curves, At least one recess is provided on the upper or lower surface of the crack-forming layer between the first notch and the second notch and the nanocrack passes through the recess.

According to one aspect of the invention the layered nanocrack structure comprises a crack-forming layer provided with a partly ring-formed opening, wherein a plate and a beam are provided in the opening. The opening surrounds the plate so that the plate is connected with a portion of the crack-forming layer outside of the opening only via the beam. The first notch comprising, the second notch and the nanocrack extending from the first notch to the second notch (are provided in the plate.

According to one aspect of the invention the a nanopore structure is provided which comprises a layered nanostructure as described above and a second crack-forming layer arranged on top of the first crack-forming layer, the second crack-forming layer comprising a second nanocrack the second nanocrack extending between a third and fourth notch arranged in the second crack-forming layer. The first nanocrack and the second nanocrack are juxtaposed so that the first nanocrack and the second nanocrack are crossing and the crossing defines a nanopore. The nanopore structure may comprise a cover layer covering all notches of the first crack-forming layer and the second crack-forming layer and is provided with an opening aligned with the nanopore, so that the only one path of fluid communication between an upper side and an lower side of the nanopore structure is through the nanopore.

The method according to the invention comprises the steps of:

providing a substrate;

deposition of a sacrificial layer on top of the substrate;

deposition of a crack-forming layer on top of the sacrificial layer;

patterning a first notch of a first diameter and a second notch of the second diameter in the crack-forming layer, wherein the first notch and a second notch are positioned a predetermined distance from each other and and arranged so that after the formation of a nanocrack a continuous opening is formed by the first notch, the second notch and the nanocrack in the crack-forming layer, the continuous opening being surrounded by a continuous structure formed in the crack-forming layer;

selectively etching, at least through the first notch and the second notch, of the sacrificial layer thereby facilitating the formation of a nanocrack in the crack-forming layer from the first notch to the second notch;

deposition of a support layer in contact with the crack-forming layer; and

patterning of an through opening in the support layer, the opening positioned so that at least a portion of the nanocrack is aligned with the opening of the support layer.

According to one aspect of the invention the support layer is provided between the sacrificial layer and the crack-forming layer.

The method may further comprise a step of forming a membrane by patterning an opening in the substrate, the opening in the substrate aligned with the opening of the support layer and extending through the thickness of the substrate from the underside and reaching the stack composed of the crack-forming layer (103), sacrificial layer and support layer. The support layer may alternatively be provided on top of the crack-forming layer and thereby forming a membrane.

According to one aspect of the invention the step of patterning the crack-forming layer comprises

patterning the crack-forming layer to form a plate and a beam in a partly ring-formed opening, and a first notch and a second notch is patterned in the plate inside the ring-formed opening, wherein each notch comprises one sharp edge and one blunt edge.

and additionally the step of selectively etching comprises the substeps of;

etching partially the sacrificial layer in the area underneath the first notch and the second notch, the etching facilitates the formation of a nanocrack in between the first notch and the second notch; and

continuing the etching so that the entire portion of the sacrificial layer underneath the plate is removed, wherein upon completion of the continuing etching the plate will be fully relaxed,

and the additional steps of:

sealing the nanocrack by providing an oxide layer in the nanocrack, this step to be taken after the partial etching of the sacrificial layer and prior to the completion of the step of continuing etching; and

selectively etching the oxide layer in the nanocrack.

According to one aspect of the invention the step of continuing the etching is arranged to release the internal stress which initate contraction of the beam so that the plate is displaced towards an anchored part of the beam, wherein the displacement of the plate, and thus the nanocrack, is proportional to the length of the beam with a proportionality constant equal to the elastic strain in the crack-forming layer, and the method comprises the further step of:

collapsing the plate onto the layers underneath the sacrificial layer by means of stiction forces by immersing in a liquid and drying.

According to one aspect of the invention a nanopore is formed, wherein a first layered nanostructure and a second layered nanostructure are formed according to the procedures described above. The method of forming a nanopore further comprising a step of arranging notches in the first layered nanostructure and second layered nanostructure so that a nanocrack extending between the notches of the first layered nanostructure will be crossing a nanocrack extending between the notches of the second layered nanostructure.

There is an advantage of the invention that it is possible to form nanocracks that are closed, or sealed, after formation featuring virtually 0 nm width by complete release-etching of the multilayered nanocrack structure. This is highly desirable in case of nanopore where a challenge is to obtain reproducible dimensions on an atomic scale. Gaps can easily be obtained from cracks that are sealed by selectively removing a native oxide which has formed in the crack.

There is an advantage with a multilayered nanocrack structure comprising a recess since when forming such a structure the total nanopore channel length is effectively reduced by the depth of the recess, which is useful for nanopore sensing devices such as in DNA sequencing that require as few nucleotides as possible to block the ionic current (ideally a single nucleotide, thereby meaning a sub-nm channel length). Furthermore, in the presence of a recess there is no need for a sharp edge at the notches, which makes the pattering part of the method of production less demanding.

It is an advantage with the invention that the widths of the nanocracks can be independent of the undercut of the release etching. This is because, the only possibility for relaxation is by relaxation of the notches and of the crack-defined surfaces, after a certain undercut is reached, these surfaces have reached maximum relaxation, and thus the parts of the crack-forming layer 103; 603 that are released are not able to relax anymore.

It is an advantage with the invention that the width of the nanocrack can be defined not only by the length of the released part of the crack-forming layer that will form a nanocrack, but also by the width of the released part. The resulting width of the nanocrack 101; 601 in case of layered nanostructure can be accurately determined by mechanical finite element modelling of the structure.

In the following, the invention will be described in more detail, by way of example only, with regard to non-limiting embodiments thereof, reference being made to the accompanying drawings.

### BRIEF DESCRIPTION OF THE DRAWINGS

FIG. 1 Illustration of a multi-layered nanocrack structure of the invention.

FIG. 2 Illustration of a nanopore structure of the invention.

FIG. 3 Illustration of a multi-layered nanocrack structure of the invention.

FIG. 4 Illustration of one embodiment of the invention.

FIG. 5 Illustration of a multi-layered nanocrack structure of the invention.

FIG. 6 Schematic illustration of a method of the invention.

FIG. 7 Schematic illustration of a method of the invention.

FIG. 8 SEM image of one multi-layered nanocrack structure of the invention.

FIG. 9 SEM image of one nanopore structure of the invention.

### DETAILED DESCRIPTION

Crack-forming layer/tensilly stressed layer: a thin film featuring internal tensile stress, or equivalently, featuring tensile elastic strain. Ideally, the stress level would be highly uniform over the entire area of the thin film on the substrate. In practice, internal stresses in thin film are of biaxial nature, such as lattice mismatch based or thermally induced stresses during and after film growth.

Notch/opening: a pattern in a layer with a depth equal to the thickness of the layer, a notch is a circular opening optionally featuring one blunt shape and one pointed form, or edge, in the multi-layered nanocrack of the invention the notches can function as:
1. crack initiator: with a notch comprising a sharp edge, a crack may nucleate at the sharp edge, which is a point of high stress concentration;
2. crack stopper: once a crack has nucleated from a notch and meets another notch, the crack may stop its propagation upon reaching the second notch, resulting in crack arrest; and
3. release opening: any notch allows the selective local removal of the sacrificial layer underneath.

Sharp edge: a surface terminating with a pointy V-like shape, such as the tail of a drop, the role of a sharp edge in a notch is to concentrate stress to initiate a crack in the crack forming layer upon release of the sacrificial layer, the sharp edge of a notch localizes crack formation selectively to other parts of a notch;

Recess/groove/V-groove: an out-of-plane stress concentration structure resulting from a local thinning of the top surface of the tensilly stressed layer; the depth of a recess is necessarily smaller than the thickness of the tensilly stressed layer so as to avoid creating an opening.

Notch pair structure/notch-only structure: a pair of notches positioned such that that a crack is initiated between the notches after release etching of the sacrificial layer underneath. A notch pair may be used alone, or in combination with a recess along where the crack is expected to propagate. A recess is necessary between notches that are circular without a sharp edge to trigger crack formation.

Gap width/widths/crack size: distance between cracked surfaces after a crack has propagated.

CMOS: complementary metal-oxide-semiconductor.

Spacer layer/sacrificial layer: a layer located underneath the tensilly stressed layer. The purpose of this layer is to provide a mechanical area of attachment for the crack forming layer onto the substrate, which can be etched selectively to the tensilly stressed layer through the notches; the local release of the tensilly stressed layer allows it be partially relax its free surfaces, which generate high stresses at the notches that trigger crack formation.

Cantilevering parts/beams: a projecting piece of material fixed at only one end, the fixed part is called the anchor;

Anchored: the fixed part of the beam, the anchor provides a mechanical fixture to the beam to lock its position on the substrate;

Sealed cracks/closed cracks: a crack wherein the first and the second side relative to the crack are in mechanical contact with one another so that the width of a sealed crack is essentially zero;

Membrane: a supporting layer that is situated below or above the crack-forming layer and at least partly are in contact with the crack-forming layer, comprising at least one opening that is at least partly aligned with the nanocrack.

Terms such as “top”, “bottom”, upper“, lower”, etc are used merely with reference to the geometry of the embodiment of the invention shown in the drawings and are not intended to limit the invention in any manner.

Generally, the invention relates to a layered structure comprising a crack-forming layer wherein a nanocrack is formed and having a continuous path of material connecting the first and the second side of the crack-forming layer. The layered nanostructure further comprises a membrane which supports the crack forming layer and provides an access to the nanocrack through a membrane opening. Such layered nanocrack structures can be used as it is, but one, or two, or more nanostructures may also be formed in the same plane and then placed on top of each-other in a juxtaposed fashion forming a nanopore in the intersection between the cracks. Such nanostructures (cracks and pores) can be useful in different applications such as electrical or optical sensing of single biomolecules, water desalination, osmotic energy harvesting, etc.

Cracks with widths in the nanometer range, i.e. nanocracks, can be obtained by creating a stress concentration in a layer on top of a substrate. The simplest approach to form a stress concentration structure in a layer is to pattern this layer to create an opening, i.e. notch, featuring one or several sharp edge(s), tip(s), corner(s), or angle(s). The sharp edge(s) of the notch will induce and localize crack formation at the point of high stress. The crack will then propagate by absorbing the mechanical energy present in the surroundings in the form of internal stresses. Upon crack formation, new crack-defined surfaces are created in the layer, which can be viewed as a type of nanofabrication. Moreover, once the cracks have formed, the crack-defined surfaces retract in opposite direction due to mechanical relaxation in the layers. It is the retraction of the crack-defined surfaces that defines the width of the crack.

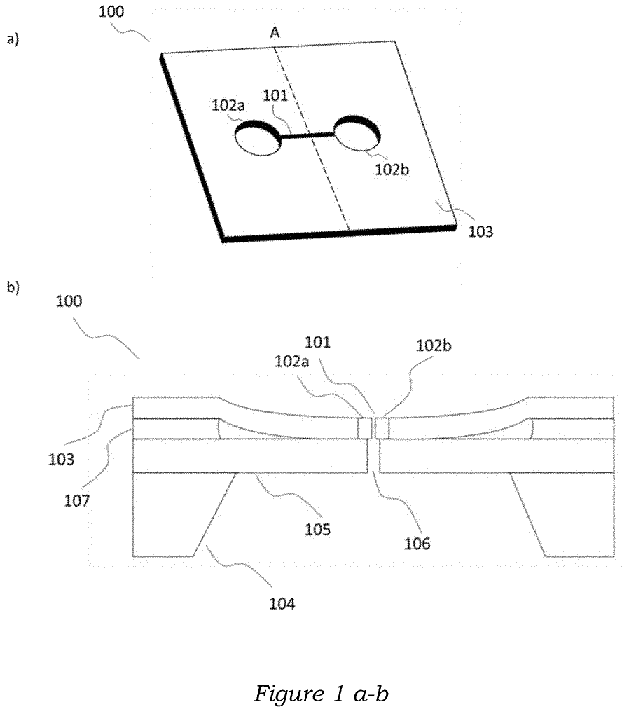

In a first aspect of the invention there is provided a layered nanostructure 100, showed in an elevated view in FIGS. 1a and in the cross-section indicated with the dashed line A in FIG. 1b. The layered nanostructure 100 comprises a_crack-forming layer 103 with a predetermined length, width and thickness, and a membrane 105 at least partly in contact with and at least partly covering the upper or lower surface of the crack-forming layer 103. The crack-forming layer 103 comprises a first notch 102a and a second notch 102b provided a distance from the first notch 102a. The first 102a and second 102b notches extend through the crack-forming layer 103. A nanocrack 101 is provided between the first notch 102a and the second notch 102b. The nanocrack 101 divides the crack-forming layer 103 into a first and second side relative the nanocrack and the crack-forming layer 103 forms a continuous structure which connects the first and the second sides of the nanocrack 101. Thereby the first notch 102a, the second notch 102b and the nanocrack 101 forms a continuous opening in the crack-forming layer 103. The membrane 105 is provided with at least one opening 106 which at least in one direction is aligned with the nanocrack 101 so that seen in the direction perpendicular to the upper surface of the crack-forming layer 103 at least a portion of the nanocrack 101 is positioned over or under the opening 106.

According to one embodiment the wherein the diameter of the first notch 102a and the diameter of the second notch 102b, respectively, is between 0.1 to 5 times the length of the nanocrack 101. The diameter should be interpreted as also valid for non-circular notches in which the diameter represents the largest diametrical distance of the circumference of the notch.

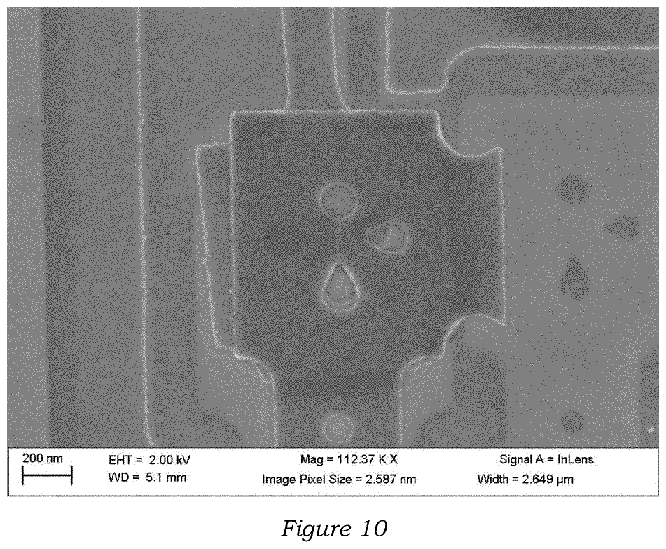

According to one embodiment of the invention, schematically shown in an elevated view in FIG. 1c and in SEM image of FIG. 9, the first notch 102a in a cross-section in the plane of the crack-forming layer 103 comprises a blunt section 106 and a sharp-edged section 107. The sharp-edge section 107 is positioned in a part of the first notch 102a facing the second notch 102b; and the nanocrack 101 extends through the crack-forming layer 103 and from the sharp edge 106 of the first notch 102a to the second notch 102b. During the process of manufacturing of the layered nanostructure the sharp-edge section 107 serves as an initiation point of the crack formation.

According to one embodiment of the invention, schematically shown in an elevated view in FIG. 1d, both the first notch 102a and the second notch 102b in a cross-section in the plane of the crack-forming layer 103 comprises of a blunt section 106a, 106b and a sharp-edged section 107a, 107b, respectively. The nanocrack 101 extends from the sharp-edge section 107a of the first notch 102a to the sharp-edge section 107b of the second notch 102b.

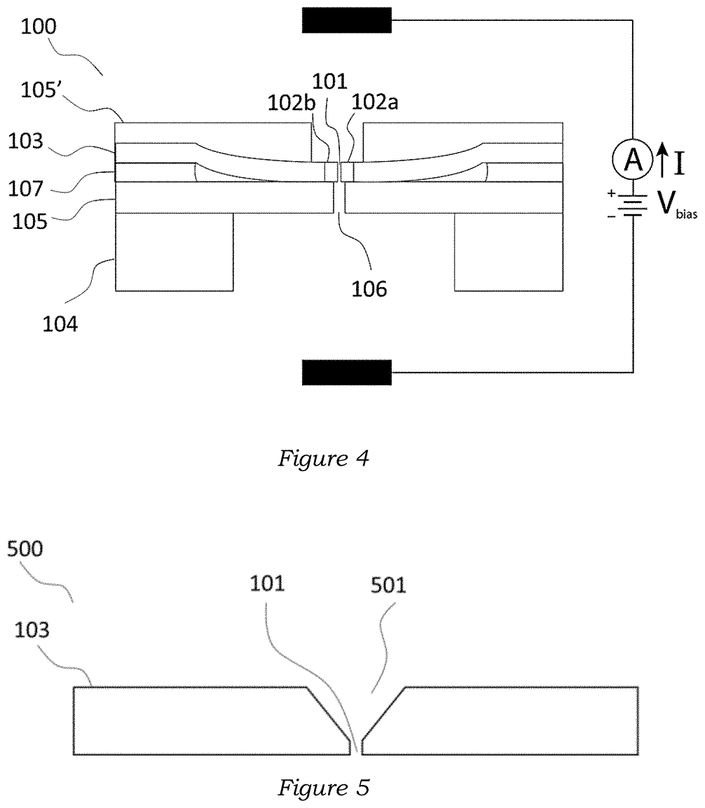

According to one embodiment of the invention, schematically shown in an elevated view in FIG. 1e both the first notch 102a and the second notch 102b in a cross-section in the plane of the crack-forming layer forms a continuous smooth curve, for example a circle or an ellipse. In order to facilitate the formation of the nanocrack 101 during the process of manufacturing a recess 501 extending from the first notch 102a and the second notch 102b, or a plurality of shorter recesses spread out between the first notch 102a and the second notch 102b, were provided in the upper surface of the crack-forming layer 103. The recess/recesses 501 may contact the notches but may also be provided a distance from the notches. The recess 501 or recesses may remain in the final layered structure. Alternatively, the recess/recesses 501 are no longer visible after the formation of the crack. According to one embodiment a recess 501 in the crack-forming layer 103 is provided at the position of the nanocrack 101, said is extending from the first notch 102a to the second notch 102b. The presence of the recess 501 results in that the depth of the nanocrack 101 is less than the thickness of the crack-forming layer 103. In such a layered nanocrack structure comprising a recess 50, the depth of the recess is 1-499 nm, preferably 1-249 nm, more preferably 1-99 nm, the structure is shown in cross section in FIG. 5. Alternatively, a plurality of recesses are provided in between the first notch 102a and the second notch 102b so that the depth of the will exhibit a variation. In another embodiment the membrane 105 is placed on top of the crack-forming layer 103. In such a structure the notches are covered with the membrane so that there can be no fluid transport through the notches in applications such as e.g. DNA sequencing.

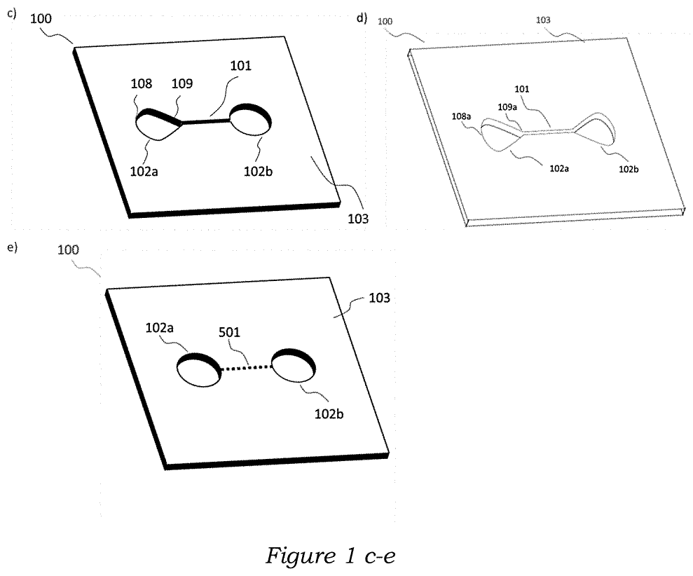

According to one embodiment, schematically illustrated in FIG. 2, the membrane 105 is placed below the crack-forming layer 103. The membrane 105 may be provided between a substrate 104 and the crack-forming layer 103 or alternatively the substrate 104 and the membrane 105 is an integral layer. According to one embodiment the membrane is placed on top of the crack-forming layer 103. In further embodiments the nano structure 100 comprises a first membrane 105 placed on top of the crack-forming layer 103 and a second membrane 105′ placed below the crack-forming layer 103.

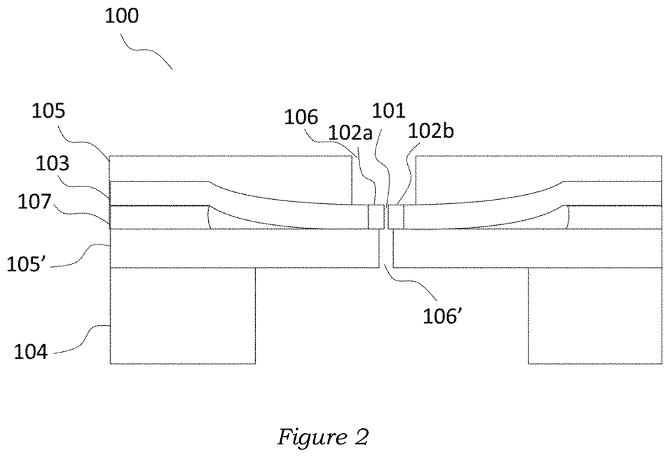

In one aspect of the invention there is provided a nanopore structure 300, schematically illustrated in an elevated view in FIG. 3 and in the SEM image of FIG. 10. Such a nanopore 301 is formed in the intersection of a first nanocrack 101a and a second nanocrack 101b, when a first and a second nanocrack structure 100 are placed on top of each other in a juxtaposed fashion so that the nanocracks 101 are crossing.

In embodiments a sacrificial layer 107 is present in the layered nanostructure 100. The sacrificial layer 107 is utilized during the process of forming the layered nano structure, which will be further discussed in the method description. The sacrificial layer 107 layer has been at least partly removed by etching, but a portion may remain in a finalized structure and may have a structural function. It is etched during the formation of the nanocrack 101. The etching results in that the crack-forming layer 103 is at least partly released, which provokes a re-distribution of stresses responsible for triggering crack formation. Furthermore, the nanocrack structure 100 may be collapsed on top of the membrane 106 by means of stiction forces.

Layered nanostructures 100 may be placed in high-density arrays such that they are placed in rows and columns, preferably aligned or misaligned to the crystal orientation of the substrate. The orientation of the nanostructures 100 can also be random with respect to the crystal orientation. When the crack-forming layer 103 comprises a single crystalline material, the orientation of the layered nanostructure 100 should preferably be such that the nanocrack 101 is formed along the crystal planes to which it is aligned.

A nanopore structure 300 according to the invention may be useful for sensing of single molecules, molecular sieving/filtration, protein detection and analysis, energy conversion, osmotic power generation, sensor ion detection, chiral recognition, responsive ionic gate etc.

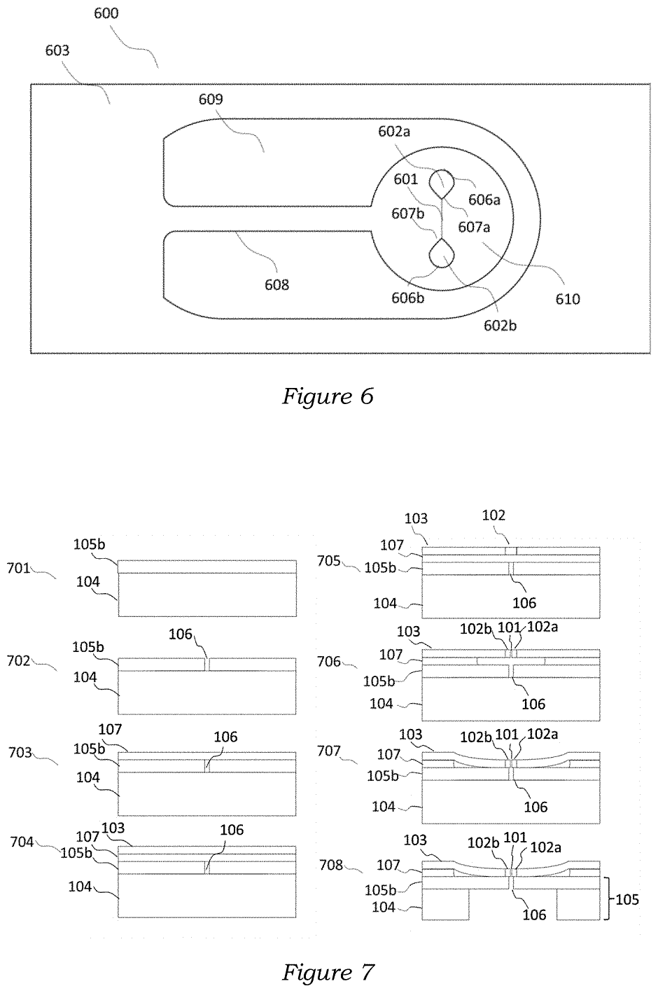

In one aspect of the invention, shown in an elevated view in FIG. 6, a layered nanocrack structure 600 comprises a crack-forming layer 603 provided with a partly ring-formed opening 609. Provided in the opening 609 is a plate 610 and a beam 608 and the opening 609 surrounds the plate 610 so that the plate 610 is connected with portion of the crack-forming layer 603 outside of the opening 609 only via the beam 608. The plate may be circular as depicted in FIG. 6, but may also have other geometrical shapes, for example quadratic or rectangular. Preferably, if the shape has corners, such corners are rounded in order to not induce unwanted cracks. The plate 610 comprises a first notch 602a and a second notch 602b, wherein the first and the second notch 602 comprises one blunt edge 606a, 606b and one sharp edge 607a, 607b each, wherein the sharp edge of the first notch 607a is directed at the sharp edge of the second notch 607b and a nanocrack 601 extending from the sharp edge 607a of the first notch 602a to the sharp edge 607b of the second notch 607b. The shortest distance between either of the first notch 602a and the second notch 602b to the opening 609 is typically in the same order as the length of the nanocrack 601. The beam 602 is typically between 100 nm and 10 μm long. Alternatively, the notches and are similar to the ones described with references to FIG. 1e and the nanocrack 601 was formed utilizing a recess or a series of recesses.

In applications where the layered nanocrack structure 100, 600 is used as electrodes, the electrodes may be electrically separated in an additional patterning step of the crack-forming layer 103 such that there is no continuous crack forming material connecting the first and the second sides of the nanocrack 101.

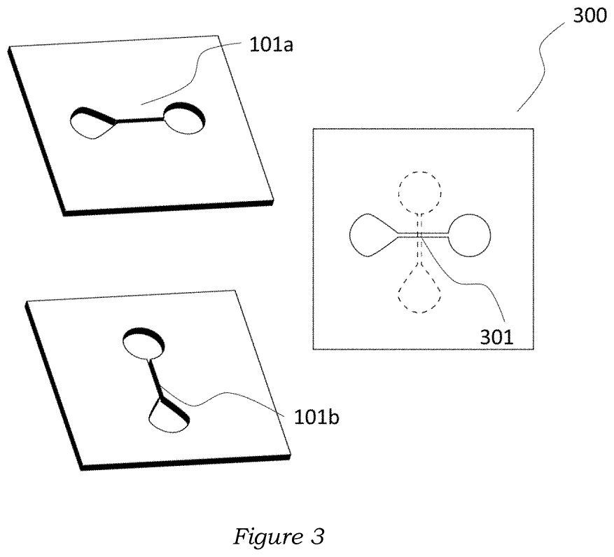

In single molecule sensing device, schematically illustrated in FIG. 4, the layered nanostructure 100; 600 and/or nanopore structure 300 are used in combination with an electrolyte with electrodes immersed on either side of the membrane 105; 105′ to electrophoretically drive single molecules through the pore and sense the changes in ionic current induced by the presence of a molecule in the pore. The only path of fluid communication between the electrolyte on either side of the nanostructure 100; 600 is via the nanocrack/nanopore.

The gap width of the nanocracks 101 of the layered crack structure 100 is 2-100 nm, preferably 2-50 nm, more preferably 2-10 nm. The length of the nanocracks 101 of the layered crack structure 100 is 5-5000 nm, preferably 5-500 nm, more preferably 5-100 nm.

The crack-forming layer 103 of the multilayered nanocrack structure 100 have a thickness of 2-500 nm, preferably 2-100 nm, more preferably 2-20 nm.

The sacrificial layer 107 of the multilayered nanocrack structure 100 have a thickness of 2-500 nm, preferably 2-100 nm, more preferably 2-25 nm.

The first notch 102a; 602a and the second notch 102b; 602b are typically of approximately the same size and a diameter between 1 and 100 times the thickness of the crack-forming layer 103, preferably between 1 and 20 times the thickness of the crack-forming layer 103, and more preferably between 1 and 10 times the thickness of the crack-forming layer 103.

The membrane 105 has a thickness of 2-500 nm, preferably 2-100 nm, more preferably 2-20 nm. The substrate 104 of the layered nanostructure 100 comprises a material from the group consisting of silicon, silicon carbide, silicon germanium, germanium, glass, quartz, sapphire, GaN, GaAs, InP, InGaAs, InAlGaAs, and polymer. In preferred embodiments the substrate 104 comprises a single crystal Si wafer comprising CMOS integrated circuits. Preferably, the substrate material is chosen so that the sacrificial layer 107 and the crack-forming layer 103 can be grown epitaxially on top of the substrate 104 without being restricted to epitaxial growth.

The sacrificial layer 107 of the layered nanostructure 100 of the invention comprises a material from the group material chosen from the group consisting of: preferably undoped or doped, Si, SiC, silicides, SiGe, Ge, GaAs, InP, InAlGaAs, InGaAs, GaP and GaN, SiN, SiO2, Al₂O₃. In preferred embodiments the sacrificial layer is single-crystalline.

The crack-forming layer 103 of the layered nanostructure 100 may preferably be made in a material chosen from the group consisting of: undoped or doped, Si, SiC, silicides, SiGe, Ge, InGaAs, GaAs, InP, InAlGaAs, GaP and GaN, SiN, SiO₂. In the case of a nanopore structure 300 the crack-forming layer 103 may preferably be an insulating material(s). In preferred embodiments the crack-forming layer is single-crystalline.

The membrane 105 of the layered nanostructure 100 may preferably be made in a material chosen from the group consisting of undoped or doped, Si, SiC, silicides, SiGe, Ge, InGaAs, GaAs, InP, InAlGaAs, GaP and GaN, SiN, SiO₂.

Method of Forming a Layered Nanostructure 100

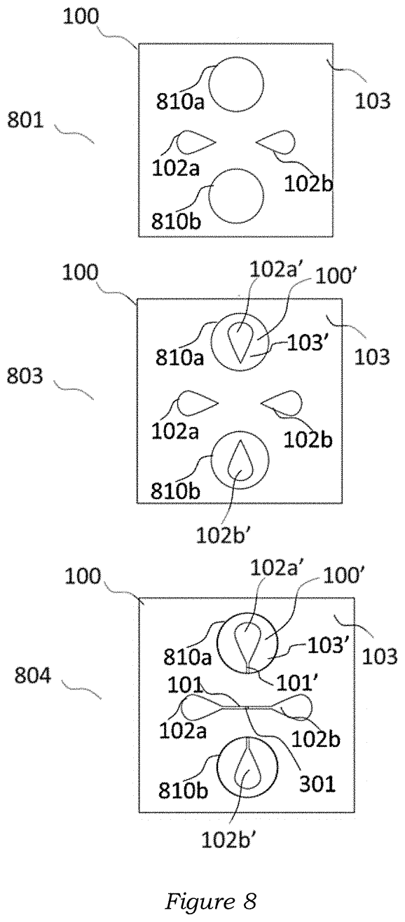

The method according to the invention to form a layered nanostructure 100 is described in the following steps, schematically illustrated in FIG. 7 showing the evolving layered nanostructure 100 in a cross-sectional view:

700: providing a substrate (not shown)

701: deposition of a support layer 105b on top of a substrate 104;

702: patterning of an opening 106 in the support layer 105 such that the opening will be aligned to the notches 102 (described in 704), wherein the opening 106 extends through the thickness of the support layer 105b;

703: deposition of a sacrificial layer 107 on of the support layer 105;

704: deposition of a crack-forming layer 103 on top of the sacrificial layer 107;

705: patterning a first 102a and a second notch 102b in the crack-forming layer 103, the patterning resulting in two notches 102 that extends through the crack-forming layer 103, wherein the notches 102 are aligned to the opening 106 in the support layer 105b;

706: selective etching, at least through the first 102a and the second notch 102b, of the sacrificial layer 107, thereby initiating the formation of a nanocrack 101 in the crack-forming layer 103 from the first notch 102a, after which the nanocrack 101 propagate until it reaches the second notch 102b where the crack propagation stops; and

707: optionally inducing the collapse of the crack-forming layer 103 onto the membrane 105 by e.g. immersing the layered nanostructure 100 in wet liquid and drying without critical point drying; and

708: structuring of the substrate 104 to form a membrane 105 by patterning an opening in the substrate which is aligned with the opening 106 of the support layer 105b and extending through the thickness of the substrate from the underside and reaching the stack composed of the crack-forming layer 103, sacrificial layer 107 and support layer 105.

The order of the steps may be changed depending on the wanted design, for example if a membrane or cover layer should be formed below or above the crack-forming layer.

Deposition can be made by atomic layer deposition (ALD), sputtering, evaporation, chemical vapor deposition (CVD), layer transfer, spray coating, spin coating, epitaxial growth etc.

Patterning can be made by masking (electron beam lithography, photolithography, nanoimprint lithography, scanning probe lithography etc) and etching (dry plasma etching, wet chemical etching, ion beam milling, sputtering, atomic layer etching etc), shadow evaporation, metal assisted etching etc.

Selective etching is achieved by selecting suitable etching parameters such as platen power, pressure, temperature, gas species, gas flow, etching chemistries etc., to etch a material with a much higher etch rate with respect to another. The highest selectivity is achieved by wet chemical etching, by choosing an adequate etching chemistry. A typical example is choosing hydrofluoric acid to etch silicon oxide with respect to silicon.

In one embodiment of the method a layered nanostructure with a ring-formed opening 600 is formed. In such a layered nanostructure with a ring-formed opening 600 the notches 602 are positioned in a plate 610 in a ring-formed opening 601 wherein the plate 610 is attached to a beam 602. Such a structure enables the formation of a nanocrack 601 with a width of less than 10 nm, preferably less than 5 nm.

The method according to the invention to forming such a structure comprises the steps of:

Step 1: a crack-forming layer 603 is patterned to form a plate 610 and a beam 608 in a partly ring-formed opening, a first notch 602a and a second notch 602b is patterned in the plate 610 inside the ring-formed opening 609, wherein each notch 602a; 606b comprises one sharp edge 607a; 606b and one blunt edge 606a; 606b;

Step 2: the sacrificial layer 107 is partially etched in the area underneath the first notch 602a and the second notch 602b, the etching initiates the formation of a nanocrack 601 in between the first notch 602a and the second notch 602b;

Step 3: the etching continues so that there is that the entire part of the sacrificial layer 107 underneath the plate 610 is removed, at this point the nanocrack 601 formed in step 2 are sealed by formation of an oxide in the nanocrack 601 and the layer comprising the plate 610 is fully relaxed, i.e. free of internal tensile stress; and

Step 4: the oxide layer in the nanocrack 601 is selectively etched forming a nanocrack 601 with a smaller width as compared with the nanocrack 601 formed in step 1, i.e. a width <10 nm.

In the methods of forming a layered nanostructure 100; 600, the notches 102a; 602a and 602b; 102b play three roles:
1. stress concentration structures to induce and localize nanocrack 101; 601 formation;
2. a stopper for the nanocrack 101; 601 propagating ; and
3. allow etching from the top of the sacrificial layer 107.

Method of Forming a Nanopore Structure 300:

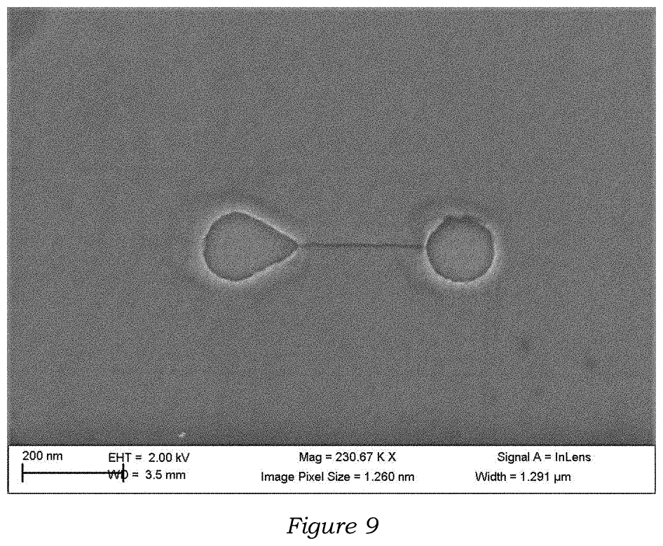

The general method to form a nanopore structure 300 is described in the following steps, schematically illustrated in an elevated view in FIG. 8:

800: (not shown) providing at least two layered nanostructures 100 and 100′ placed on top of each other, wherein each layered nanostructure 100 comprises a at least, a sacrificial layer 107, and a crack-forming layer 103;

801: patterning of a first 102a and a second notch 102b, and a first 810a and a second circular opening 810b in the first layered nanostructure 100 that is placed on top, the notches 102a; 102b and the circular openings 810a; 810b extending through the crack-forming layer 103 of the first layered nanostructure 100, wherein the circular openings 810a; 810b are positioned so that they will be positioned above the notches 102a and 102b of the second layered nanostructure 100′;

802: (not shown) selective etching of the sacrificial layer 107 of the first layered nanostructure 100, thereby providing access to the crack-forming layer 103′ of the second layered nanostructure 100′;

803; pattern of a first 102a′ and a second 102b′ notch in the second layered nanostructure 100′ through the first 810a and second circular opening 810b;

804: selective etching of the crack-forming layer 103′ of the second layered nanostructure 100′, thereby forming a first 101 and a second 101′ nanocrack extending from the first 102a; 102a′ to the second notch 102b; 102b′, forming a nanopore 301 in the intersection between the first 101 and the second nanocrack 101′;

805: (not shown) optionally collapse the first 100 and the second 100′ layered nanostructure on to each other; and

806: (not shown) optionally deposit a cover layer covering all openings except the nanopore 301, in this way all other flow paths are obstructed.

In one embodiment of the method there is a method of displacing a nanocrack 101

First a layered nanostructure with a ring-formed opening 600 is formed as described in Steps 1-4 above, followed by:

Step 5: the release of the internal stress initates contraction of the beam 608 so that the plate 610 is displaced towards the anchored part of the beam 608, wherein the displacement of the plate 610, and thus the nanocrack 601, is proportional to the length of the beam 608 with a proportionality constant equal to the elastic strain in the crack-forming layer 603.

Step 6: the plate 610 is collapsed onto the material underneath the sacrificial layer 107 by means of stiction forces by immersing in a liquid and drying.

The embodiments described above are to be understood as illustrative examples of the system and method of the present invention. It will be understood that those skilled in the art that various modifications, combinations and changes may be made to the embodiments. In particular, different part solutions in the different embodiments can be combined in other configurations, where technically possible.

1. A layered nanostructure comprising:
a crack-forming layer with an upper and lower surface;
a membrane at least partly in contact with and at least partly covering the upper or lower surface of the crack-forming layer;
a first notch and a second notch provided in and extending through the crack-forming layer, the first notch provided at a distance from the second notch;
a nanocrack provided between the first notch and the second notch, the nanocrack dividing the crack-forming layer into a first and second side relative the nanocrack; and
a through opening arranged in the membrane, the opening positioned so that at least a portion of the nanocrack is aligned with the opening of the membrane,
wherein a continuous opening is formed by the first notch, the second notch and nanocrack in the crack-forming layer, the continuous opening being surrounded by a continuous structure formed in the crack-forming layer, the continuous structure connecting the first and the second sides of the nanocrack.

2. The layered nanostructure according to claim 1, wherein the diameter of the first notch and the diameter of the second notch, respectively, is between 0.1 to 5 times the length of the nanocrack.

3. The layered nanostructure according to claim 1, wherein the membrane is provided with at least one opening which at least in one direction is aligned with the nanocrack so that at least a portion of the nanocrack is positioned over or under the opening.

4. The layered nanostructure according to claim 1, wherein the first notch in a cross-section in the plane of the crack-forming layer comprises a blunt section and a sharp-edged section;
the sharp-edge section is positioned in a part of the first notch facing the second notch; and
wherein the nanocrack extends from the sharp-edged section of the first notch to the second notch.

5. The layered nanostructure according to claim 4, wherein the second notch in a cross-section in the plane of the crack-forming layer comprises of a blunt section and a sharp-edged section, and wherein the nanocrack extends from the sharp-edge section of the first notch to the sharp-edge section of the second notch.

6. The layered nanostructure according to claim 1, wherein nanocrack extends in the direction representing the shortest distance between the first notch and the second notch.

7. The layered nanostructure according to claim 1, wherein in a cross-section in the plane of the crack-forming layer the first notch forms a smooth continuous curve and the second notch forms a smooth continuous curve, and at least one recess is provided on the upper or lower surface of the crack-forming layer between the first notch and the second notch and the nanocrack passes through the recess.

8. The layered nanostructure according to claim 5, wherein the layered nanocrack structure comprises a crack-forming layer provided with a partly ring-formed opening, wherein a plate and a beam are provided in the opening and the opening surrounds the plate so that the plate is connected with a portion of the crack-forming layer outside of the opening only via the beam, and wherein the first notch comprising the blunt edge and the second notch comprising the blunt edge and the sharp edge and the nanocrack extending from the first notch to the second notch are provided in the plate.

9. A nanopore structure, comprising the layered nanostructure according to claim 1, comprising the first crack-forming layer comprising the first nanocrack, the nanopore structure further comprising a second crack-forming layer arranged on top of the first crack-forming layer, the second crack-forming layer comprising a second nanocrack, the second nanocrack extending between a third and fourth notch arranged in the second crack-forming layer, wherein the first nanocrack and the second nanocrack are juxtaposed so that the first nanocrack and the second nanocrack are crossing and the crossing defining a nanopore.

10. The nanopore structure according to claim 9, further comprising a cover layer covering all notches of the first crack-forming layer and the second crack-forming layer and provided with an opening aligned with the nanopore, so that the only one path of fluid communication between an upper side and an lower side of the nanopore structure is through the nanopore.

11. A method of producing a layered nanostructure, the method comprising:
providing a substrate;
deposition of a sacrificial layer on top of the substrate;
deposition of a crack-forming layer on top of the sacrificial layer;
patterning a first notch of a first diameter and a second notch of the second diameter in the crack-forming layer, wherein the first notch and a second notch are positioned a predetermined distance from each other and the first and second diameter and arranged so that after the formation of a nanocrack a continuous opening is formed by the first notch, the second notch and the nanocrack in the crack-forming layer, the continuous opening being surrounded by a continuous structure formed in the crack-forming layer;
selective etching, at least through the first notch and the second notch, of the sacrificial layer, thereby facilitating the formation of a nanocrack in the crack-forming layer from the first notch to the second notch;
deposition of a support layer in contact with the crack-forming layer; and
patterning of an through opening in the support layer, the opening positioned so that at least a portion of the nanocrack is aligned with the opening of the support layer.

12. The method according to claim 11, wherein the support layer is provided between the sacrificial layer and the crack-forming layer.

13. The method according to claim 11, further comprising a forming a membrane by patterning an opening in the substrate, the opening in the substrate aligned with the opening of the support layer and extending through the thickness of the substrate from the underside and reaching the stack composed of the crack-forming layer, sacrificial layer and support layer.

14. The method according to claim 11, wherein the support layer is provided on top of the crack-forming layer and thereby forming a membrane.

15. The method according to claim 11, wherein said patterning the crack-forming layer comprises:
patterning the crack-forming layer to form a plate and a beam in a partly ring-formed opening, and a first notch and a second notch is patterned in the plate inside the ring-formed opening, wherein each notch comprises one sharp edge and one blunt edge.

16. The method according to claim 15, wherein said selectively etching comprises:
etching partially the sacrificial layer in the area underneath the first notch and the second notch, the etching facilitates the formation of a nanocrack in between the first notch and the second notch; and
continuing the etching so that the entire portion of the sacrificial layer underneath the plate is removed, wherein upon completion of the continuing etching the plate will be fully relaxed, and
wherein the method further comprises:
sealing the nanocrack by providing an oxide layer in the nanocrack, after the partial etching of the sacrificial layer and prior to the completion of the step of continuing etching; and
selectively etching the oxide layer in the nanocrack.

17. The method according to claim 16, wherein said continuing the etching is arranged to release the internal stress which initates contraction of the beam so that the plate is displaced towards an anchored part of the beam, wherein the displacement of the plate, and thus the nanocrack, is proportional to the length of the beam with a proportionality constant equal to the elastic strain in the crack-forming layer, and the method further comprises:
collapsing the plate onto the layers underneath the sacrificial layer by means of stiction forces by immersing in a liquid and drying.

18. A method of forming a nanopore, wherein a first layered nanostructure and a second layered nanostructure are formed according to the method of claim 11, the method of forming a nanopore further comprising arranging notches in the first layered nanostructure and second layered nanostructure so that a nanocrack extending between the notches of the first layered nanostructure will be crossing a nanocrack extending between the notches of the second layered nanostructure.
